(19) United States
(12) Patent Application Publication
(10) Pub. No.: US 2015/0202037 A1
(43) Pub. Date: Jul. 23, 2015

(54) Vascular Filter for Protection During Surgery

(71) Applicant: Gregory Sullivan, Quincy, MA (US)

(72) Inventor: Gregory Sullivan, Quincy, MA (US)

(21) Appl. No.: 14/602,578

(22) Filed: Jan. 22, 2015

Related U.S. Application Data
(60) Provisional application No. 61/930,667, filed on Jan. 23, 2014.

(51) Int. Cl. A61F 2/01

(52) U.S. Cl. CPC A61F 2/013; A61F 2002/011; A61F 2/95

(57) ABSTRACT

A filtration system to collect debris in the vascular system and/or other systems and a method for using such filter. An internally disposed apparatus for inside a fluid conduit during a medical procedure which includes a filtering membrane and a frame connected to the membrane. In one embodiment of the filtration system, a filter may generally include a membrane, a frame, and a rod.

### CROSS REFERENCE TO RELATED APPLICATION

This applications takes priority from and claims the benefit of U.S. Provisional Patent application Ser. No. 61/930,667 filed on Jan. 23, 2014, the contents of which are herein incorporated by reference.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

1. Field of the Invention

Embodiments of the present invention relate to a vascular filter for protection during surgery. In certain embodiments, the present invention relates specifically to systems and methods involving angioplasty and/or stenting to protect against loose embolic material or other debris.

2. Description of the Related Art

Angioplasty and stenting are performed to remove obstructions or blockages in arteries and thereby alleviate life-threatening conditions. The procedures may result in a fracturing or disintegration of the obstructing material and if the resulting particles, or debris, were permitted to flow downstream within the circulatory system, they can cause blockages in smaller arteries, or their microscopic branches termed the microcirculation, downstream of the treatment site. The result can be new life-threatening conditions, including stroke.

Various systems and techniques exist to remove debris from the circulatory system, including temporarily obstructing the artery by means such as a balloon and then suctioning debris and blood from the treatment site. While such techniques can effectively solve the problem stated above, they require that blood flow through the artery be obstructed, causing complete cessation or at least a substantial reduction in blood flow volume, during a time period which can be significant for organ or cell survival.

Filters have also been used to collect debris in the vascular system. The filters are generally inserted before the procedure to trap debris and then closed and removed with the trapped debris after the procedure. Multiple problems exist for filters in use today. One problem is that debris can escape a filter from the proximal end (opening) when the filter is closed for removal.

Another issue that arises focuses on debris that may be squeezed through the holes of the filter when the filter is closed. Another problem is that the size and/or inflexibility of the filter prevent the filters from being used in distal sections or peripheral arteries of the body. For example, a filter used in the carotid artery is unable to be used in a peripheral artery located in the foot. Another problem is that filters are fixed as to make it impossible for an additional device to enter the filter for additional treatment such as flushing or suction. Another problem is that the length and/or rigidity of the filters cause the filter poorly fit in strong bent arteries and thus be deformed or have gaps between the wall of the artery and the filter.

Another problem is that the length of filters cause the filters to be placed further away from the lesion. Another problem is that unwanted movement by the person holding the guideline for the filter may cause an unwanted influence in orientation or geometry of the filter. Another problem is that the membrane of the filter is thin and fragile and may tear during use, thus preventing from a sufficient number of holes being made in the membrane for filtering.

### SUMMARY OF THE INVENTION

The instant apparatus and system, as illustrated herein, is clearly not anticipated, rendered obvious, or even present in any of the prior art mechanisms, either alone or in any combination thereof. A versatile system, method and series of apparatuses for creating and utilizing a vascular filter for protection during surgery. Thus the several embodiments of the instant apparatus are illustrated herein.

It is an object of the instant system to introduce a series of systems and methods involving angioplasty and/or stenting which protect against loose embolic material and other debris.

It is an object of the instant system to introduce a system including a filter with a membrane, a frame and a rod and the filter includes a self-expanding cylindrical nitinol stent.

It is an object of the instant system to introduce a system utilizing a distally disposed ring that slides on the rod at a distal end.

It is an object of the instant system to introduce a system utilizing a vascular filter for protection during surgery.

There has thus been outlined, rather broadly, the more important features of the versatile vascular filter for protection during surgery embodiments in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

Advantages of the present invention and better understanding will be apparent from the following detailed description of exemplary embodiments thereof, which description should be considered in conjunction with the accompanying drawings, in which:

FIG. 1 is an embodiment of a filter in various geometrical orientations;

FIG. 2a is an example membrane of the filter in FIG. 1;

FIG. 2b is an example membrane in FIG. 2a from a different orientation;

FIG. 3 shows one embodiment of the frame of the filter in FIG. 1;

FIG. 4a is an example strut connectors of the frame in FIG. 3;

FIG. 4b shows a front view of the strut connector in FIG. 4a;

FIG. 4c shows a side view of the strut connector in FIG. 4a;

FIG. 4d shows a top view of the strut connector in FIG. 4a;

FIG. 5a is an example of the frame in FIG. 3 while the filter is in a collapsed position;

FIG. 5b is an example of the frame in FIG. 5a while the filter is in an open position;

FIG. 6 is a filter in FIG. 1 during removal of the filter;

FIG. 7 is a filter in FIG. 1 while a suction device is inserted into the filter;

FIG. 8 is a flowchart of an example method for inserting the filter in FIG. 1;

FIG. 9 is a flowchart of an example method for removing the filter in FIG. 1;

FIG. 10 illustrates a side perspective view of a pair of filter configurations with fiber reinforced membranes;

FIG. 11 illustrates an additional side perspective view of a filter configuration with fiber reinforced membranes;

FIG. 12 illustrates a side perspective view of a suction device entering the filter configuration with fiber reinforced membranes;

FIG. 13 illustrates a deployment and inflation diagram for one embodiment of the instant system and accompanying apparatuses; and,

FIG. 14 illustrates a mesh stent loaded inside a catheter for one embodiment of the instant system and accompanying apparatuses.

### DETAILED DESCRIPTION OF THE PREFERRED EMBODIMENTS

Embodiments of the present series of apparatuses, systems and interrelated methods pertain to a vascular filter for protection during surgery. In certain embodiments, the present series of apparatuses, systems and interrelated methods relate specifically to systems and methods involving angioplasty and/or stenting to protect against loose embolic material or other debris. Throughout the description, for the purposes of explanation, numerous specific details are set forth in order to provide a thorough understanding of the present invention. It will be apparent, however, to one skilled in the art that the present invention may be practiced without some of these specific details. In other instances, well-known structures and devices are shown in generic form to avoid obscuring the underlying principles of the present invention.

### Filter

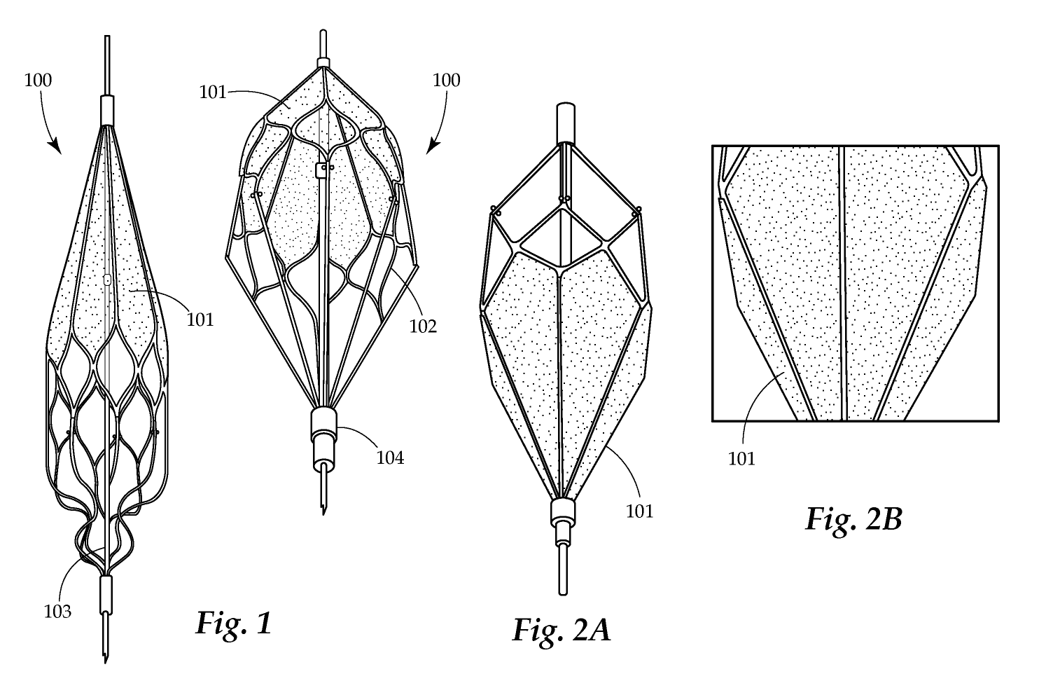

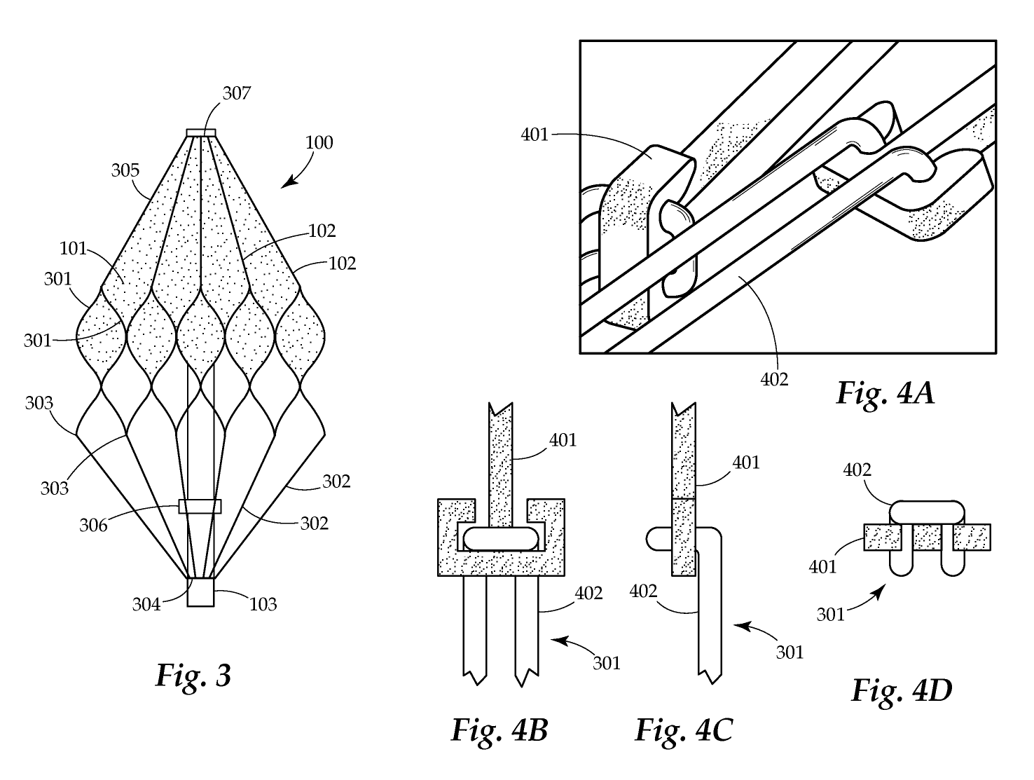

FIG. 1 is a photograph of an embodiment of a filter 100 various geometrical orientations. The filter 100 generally comprises: a membrane 101; a frame 102; and a rod 103. In one embodiment, the filter 100 is based on a 7 mm long self-expanding cylindrical nitinol stent, acting as the frame 102 that keeps the filter 100 in position. In another embodiment, the length may range from 0.1 mm to 20 mm or any range within this range. Referring back to FIG. 1, the frame 102 is connected to the membrane 101. In one embodiment, the tip of the membrane 101 is connected to a distal ring (307 of FIG. 3) that slides on the rod 103 at a distal end. Distal is the end farthest away from the lesion where the device is to enter. On the proximal end, the rod is connected to the frame 102. Proximal is the end opposite the distal end and closest to the lesion where the device enters.

The rod 103 may have a flexible, radio opaque distal tip to allow maneuverability within a blood vessel. In one embodiment, the rod is 0.35 mm in diameter and a guidewire for the person inserting the filter 100. In another embodiment, the rod diameter ranges from 0.01 mm to 15 mm or any range within this range. The filter 100 may be housed by a sheath 104 (e.g., a tube) during insertion. In one embodiment, the tube 104 is used during the procedure as a suction or flushing device in order to remove debris from the filter 100. Furthermore, in one embodiment the system may utilize a 0.018 mm guidewire.

### Membrane

FIGS. 2a and 2b are photographs of an example membrane of the filter in FIG. 1. In one embodiment, the membrane 101 is thin and pliable. The membrane 101 may be, but is not limited to, a mesh (e.g., wire mesh), paper filter, a perforated plastic, or any other opaque material with holes or porous material to allow the passage of fluid. The membrane may have a plurality of holes in order to filter blood or other fluids and trap debris. In one embodiment, the membrane includes approximately 1800 holes or apertures, wherein the diameter of each hole may be approximately 110 microns. In another embodiment, the number of holes may range from 1 to 10,000 or any range within this range. Also in another embodiment, the diameter of the holes may range from 30 microns to 500 microns or any range within this range. The holes may be cut into the membrane by a laser or other cutting devices.

### Frame

FIG. 3 shows one embodiment of the frame 102. In one embodiment, the frame 102 generally comprises: struts 301; free moving fibers 302; strut connectors 303 to connect struts 301 to free moving fibers 302 and/or reinforcement fibers 305; guide ring 304 to connect the free moving fibers 302 to the rod 103; and reinforcement fibers 305 connected to the struts 301 (by strut connector 303) and the membrane 101.

In one embodiment, the free moving fibers 302 are fine ultra high molecular weight polyethylene fibers with high flexibility and extreme tensile strength and a thickness of approximately 50 microns. When expanded, the strut section 301 fits to the artery wall and leave the proximal entrance of the filter 100 open. In one embodiment, the struts are a shape memory alloy, such as nitinol, and the frame 102 is opaque to radio signals. Radio opacity of the frame 102 may be enhanced by a coating. For example, a gold-coating of 3 microns thickness may be applied to the frame 102 so that the status of deployment of the filter 100 is well visible on X-ray.

In one embodiment, the dimensions of the frame are a length of 7 mm and an external diameter of 0.7 mm collapsed and 7 mm expanded. In another embodiment, the external diameter may range from 0.1 mm collapsed to 20 mm expanded or any range within this range. The rod 103 may include a mechanical stop 306 to engage the guide ring 304 of the frame 102 or the distal ring (307 of FIG. 3) attached to the membrane 101 at the distal end when the rod 103 is moved far enough forward or backward. Therefore, the rod 103 may move in any direction without influencing, interfering, or changing the geometry and/or position of the filter 100, as long as the stop 306 on the rod 103 stays within the range of free movement between the two rings 304 and 307. The mechanical stop 306 does not require mechanical interaction and therefore allows the frame 102 to assume an ideal wall apposition, even if the filter 100 is placed in a strongly curved artery.

In one embodiment, the guide ring 304 allows inclusion of an additional device, such as a suction tube, into the filter 100 in order to suction it empty and thus prevent a pile-up of debris. In another embodiment, the free moving fibers 302 allow the insertion of such device. FIG. 7 illustrates a suction device being inserted into the filter 100. In one embodiment, the sheath 104 acts as the suction tube. In another embodiment, the suction tube that fits in the guide ring 304 beside the rod 103 or between the free moving fibers 302. Cleaning of the filter 100 allows the filter 100 to be left in place for a longer period without the problem of full occlusion, or to use it in cases where extreme amounts of debris are expected.

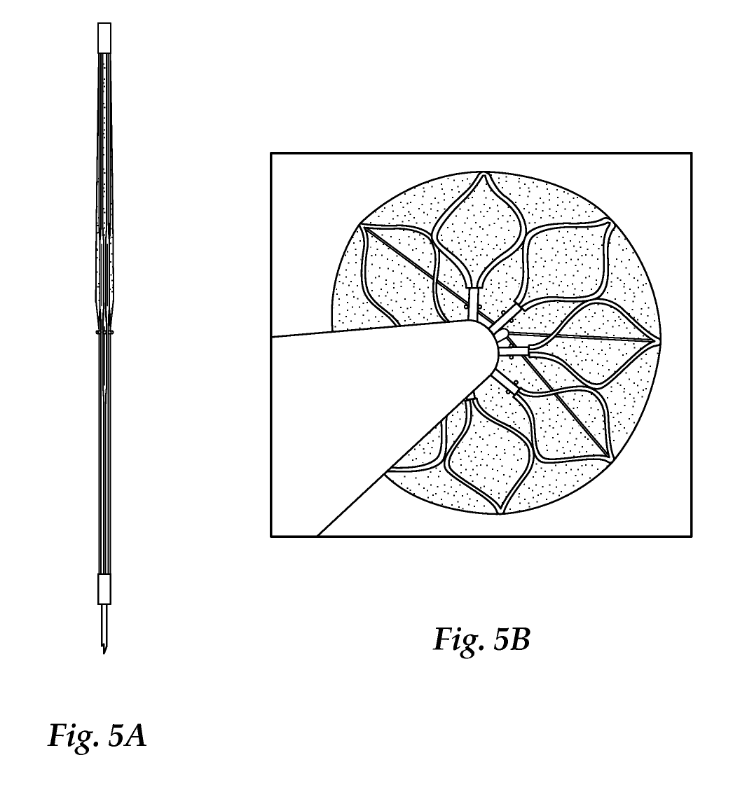

One embodiment of attaching the struts 301 to free moving fibers 302 is the strut connectors 303. FIGS. 4a-d illustrate an example strut connector 303. In one embodiment, the strut connector is an anchor shaped end (401 of FIGS. 4b-d) of a strut 301 attached to a bend (402 of FIGS. 4b-d) of a free moving fiber 302. Other embodiments of attaching the struts 301 to the free moving fibers 302 include, but are not limited to, tying the fiber 302 to the strut 301 and adhesives (e.g., glue).

As previously stated, in one embodiment, the frame 102 includes reinforcement fibers 305 connected to the membrane 101. Since the membrane may be thin and pliable, reinforcement fibers 305 are connected to the membrane 101 in order to prevent cracking or separation of the membrane 101 from the frame 102. In one embodiment, the fibers 305 are wrapped around the frame struts 301 and then embedded into the membrane 101 to prevent accidental detachment from the frame 102. An example connection of the reinforcement fiber 305 to the strut 301 is illustrated in FIGS. 4a-d. In one embodiment, the reinforcement fibers 305 run from a distal ring 307 connected to the membrane 101 (described above), loop around the end of the strut 301, and connect back to the distal ring 307. Alternatively or in addition, an adhesive may be used to attach the membrane 101 and/or the struts 301 to the reinforcement fibers 305. One example adhesive includes polyurethane, which may be applied through a dipping process. In another embodiment, the reinforcement fiber 305 is woven through the holes of the membrane 101.

The reinforcement fibers 305 may be from a multitude of materials, but one example reinforcement fiber is a fine multifilament fiber of high molecular weight polyethylene. In one embodiment, the tensile strength of the fiber exceeds 3000 mega Pascal (MPa) and flexible. In one embodiment, the flexibility of the fiber is limited in the length direction such that the maximum increase in length is approximately 3%. The fibers retain the properties of flexibility and tensile strength after thousands of cycles of use. The reinforcement fibers 305 also allow the membrane 101 to wrap around debris without squeezing during closure of the filter 100. The reinforcement fibers 305 also receive the tensile stress from the rod 103 when removing the filter 100 from a sheath 104 and pull the membrane 101 into place.

The size, flexibility, and expandability of the filter 100 allow for the filter 100 to be used in multiple size blood vessels, including large arteries, such as the carotid artery or aorta, to peripheral arteries, such as those found in distal limbs of the body (e.g., the foot or hand).

### Insertion of Filter

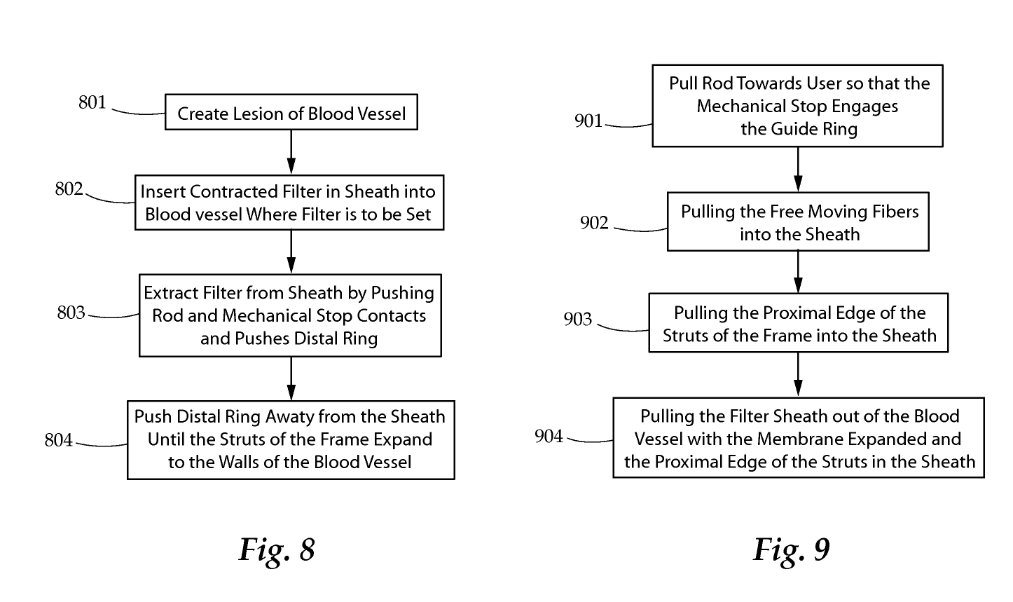

FIG. 8 is a flowchart of an example method of inserting the filter 100 into a blood vessel of the body. Beginning at 801, a lesion is created in the blood vessel. In one embodiment, the blood vessel is punctured by a hollow wire. Proceeding to 802, the sheathed filter 100 (e.g., in a tube) is inserted into the blood vessel far enough so as to set the filter 100 in the desired place within the blood vessel. Once the sheath 104 is inserted the desired distance into the blood vessel, the filter 100 is extracted from the sheath 104 in 803. In one embodiment, the filter 100 is extracted by pushing the rod 103 so that the mechanical stop 306 (FIG. 3) engages the distal ring 307 and pushes the ring 307 out of the sheath 104. The distal ring 307 pulls the reinforcement fibers 305 out of the sheath 104, which pulls the membrane 101 and struts 301 out of the sheath 104. Once the struts 301 are pulled out of the sheath 104, the struts 301 of the frame 102 expand to fit to the walls of the blood vessel in 804.

FIG. 5a illustrates the struts 301 of the frame while in the sheath 104. The frame 104 is compressed into a small diameter for easy insertion into the blood vessel. FIG. 5b illustrates the struts 301 when removed from the sheath 104. The frame 104 expands and spreads the membrane 101 in order to filter the blood vessel for debris.

### Removal of Filter

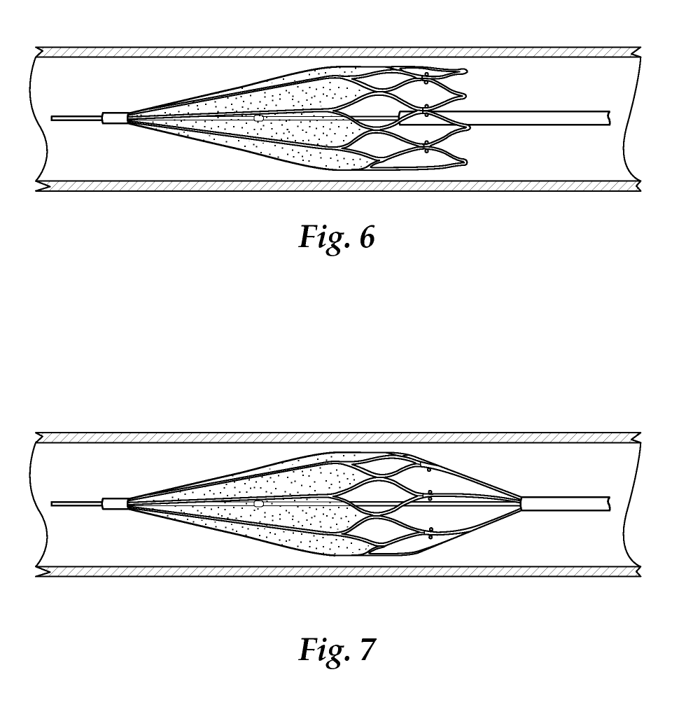

In one embodiment, the strut section of frame 102 of the filter 100 may be collapsed without changing the shape of the membrane 101. FIG. 9 is a flowchart of an example method for removing a filter 100. Beginning in 901, the rod is pulled towards the user and the mechanical stop 306 of the rod 103 engages the guide ring 304 of the frame 102. The guide ring 304 is then pulled into the sheath 104. Pulling the guide ring 304 into the sheath 104 pulls the free moving fibers 302 connected to the guide ring 304 into the sheath 104 (902). Once the free moving fibers 302 are in the sheath 104, the free moving fibers pull the struts 301 at the strut connectors 303 so as to radially compress the proximal edge of the strut section and pull the section into the sheath 104 (903). The proximal fibers pulling at the strut connectors 303 of the struts 301 creates a conical section in the frame. FIG. 6 illustrates the canonical form of the frame during removal of the filter.

FIG. 6 also illustrates that, while the struts 301 are pulled further inside the sheath 104, the membrane 101 is still in its fully deployed state and gives full distal protection. When the strut section is compressed, the gaps between the struts 301 of the frame 102 close. In one embodiment, the closing frame acts as a cap that closes the proximal entrance of the filter, thus preventing any loss of captured debris, and acts as an additional filter. Referring back to FIG. 9, the filter is extracted with the membrane 101 expanded and the proximal edge of the struts 301 sheathed. Since the membrane is expanded during extraction, no debris is able to escape from the filter during removal.

Embodiments of the invention may include various processes or components as set forth above. It will be apparent to one skilled in the art that not all components or processes are required, and the processes described for insertion and extraction of the filter may be in different order. In addition, while the filter has been described in terms of being used in the vascular system, other uses of the filter exist.

For example, the filter may be used in various piping not associated with the human body, the gastrointestinal system, the respiratory system, and/or other fluid conduits. In another example, while the reinforcement fibers are shown as lying longitudinally and approximately parallel to the rod, the reinforcement fibers may be any network or pattern, including a randomly oriented network. In another example, while the membrane is described as being stretched like an umbrella, reinforcement fibers may be fused with or be a shape memory alloy (e.g., nitinol) so as to control the shape the membrane. In another example, expandable or deformable frames are used.

In another example, while the filter is described as being attached, other devices may be attached to the sheath or rod. In an embodiment, additional proximal fibers are attached to such devices. Examples include removable temporary stents, occlusion devices, grafts, valves, clips, retrieval bags, inflatable members, devices for body tissue replacement and delivery platforms for drugs, radiation or gene therapy.

In another example, while a sheath is described as a tube, a sheath may include, but is not limited to, a ring to compress the frame, a latch attached to the struts to lock the frame in a compressed state, an at least one Micro Electrical Mechanical (MEM) motor or other motor to open and close the frame, or the frame being a piezoelectric material in order to compress when an electric current is introduced. In another example, while the frame is described as including a stent structure, the frame may alternatively include a plurality of crossbeams attached to the rod in order to open the membrane for filtering. In another example, while the strut connector is described as including an anchor shape structure, many shapes may be utilized, including a loop or a hook.

In another example, while a mechanical means is described for inserting, opening and removing the filter, the filter may be opened by other means including, but not limited to, fluid pressure to open the membrane for filtering or pressure from the artery wall to trigger opening of the filter. In a further example, while a radio opaque material is described for coating the frame for tracking the location of the filter, other materials may coat or be embedded in the material of the frame or filter including, but not limited to, a slight radioactive material that emits energy (e.g., through doping of the metal or coating) or a photo luminescent material to reflect light shined on the filter. In another example, while fibers are described as being polyethylene, other materials including metal, textiles, glass, or plastics may be used. In addition, while fibers are described, other means including threads or rope may be used. In another embodiment, while debris is described as embolic material, debris may be any material unwanted (e.g., foreign object) and thus to be removed.

In another example, removing the filter while the membrane is open is described, other removal means may occur including the membrane being closed and/or compressed to wrap around trapped debris during removal. In another example, while rings are described for engaging a stop, other engagement means may exist including, but not limited to, a hook, nub, protrusion, or friction surface.

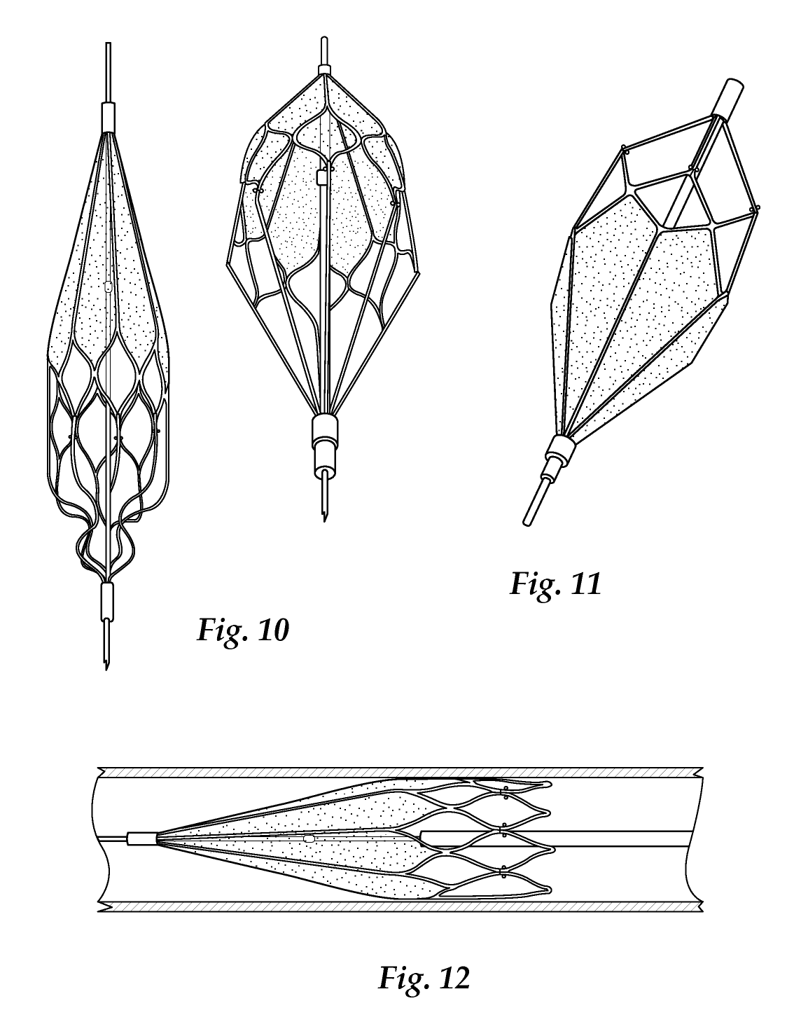

In additional embodiments, much like FIG. 1, FIG. 10 illustrates a side perspective view of a pair of filter configurations with fiber reinforced membranes. Additionally, FIG. 11 illustrates an additional side perspective view of a filter configuration with fiber reinforced membrane. And, FIG. 12 illustrates a side perspective view of a suction device entering the filter configuration with fiber reinforced membranes.

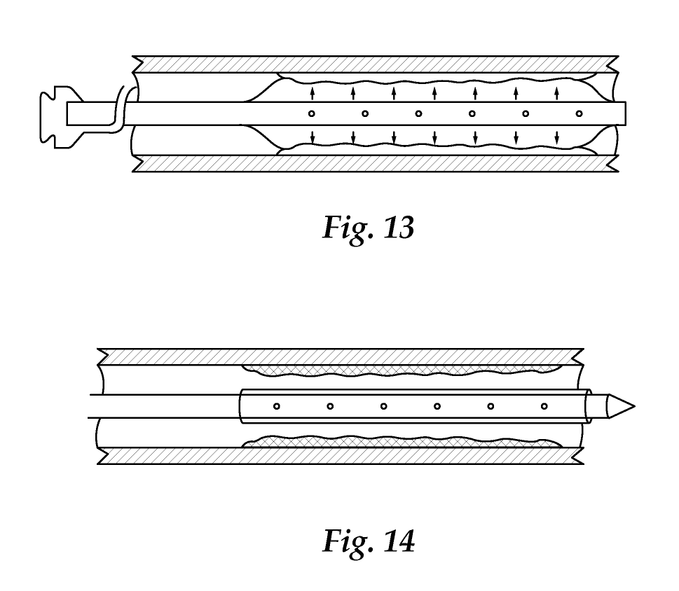

FIG. 13 illustrates a deployment and inflation diagram for one embodiment of the instant system and accompanying apparatuses. Moreover, FIG. 14 illustrates a mesh stent loaded inside a catheter for one embodiment of the instant system and accompanying apparatuses.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein. All of the herein described embodiments are intended to be within the scope of the invention herein disclosed.

What is claimed is:

1. An apparatus for inside a fluid conduit during a procedure, comprising:
a membrane to filter a fluid of the fluid conduit; and
a frame connected to the membrane at a distal end and extended to hug the walls of the fluid conduit, the frame to be compressed at a proximal end while the distal end remains extended;
wherein the distal end of the frame extends the membrane to the walls of the fluid conduit; and
wherein the membrane remains extended during the removal of the apparatus from the fluid conduit.

2. The apparatus for inside a fluid conduit during a procedure of claim 1 further comprising a rod.

3. The apparatus for inside a fluid conduit during a procedure of claim 2, wherein the membrane comprises a tip member, a distal ring and a distal end, wherein the tip member of the membrane is connected to a distal ring that slides on the rod at a distal end.

4. The apparatus for inside a fluid conduit during a procedure of claim 1, wherein the distal end is disposed farthest away from the lesion where the apparatus is to enter.

5. The apparatus for inside a fluid conduit during a procedure of claim 2, wherein the rod further comprises a proximal end and the proximal end of the rod is connected to the frame.

6. The apparatus for inside a fluid conduit during a procedure of claim 5, wherein the proximal end of the rod is disposed to be closer to the lesion where the apparatus enters the body than the distal end.

7. The apparatus for inside a fluid conduit during a procedure of claim 1 further comprising a self-expanding cylindrical nitinol stent.

8. The apparatus for inside a fluid conduit during a procedure of claim 1, wherein the frame comprises:
a stent section in communication with the walls of the fluid conduit; and
a plurality of reinforcement fibers connected to the stent section and the membrane, wherein the reinforcement fibers are disposed to prevent the membrane from breaking.

9. The apparatus for inside a fluid conduit during a procedure of claim 1, wherein the conduit comprises an artery.

10. A method to prevent a quantity of debris from escaping an apparatus in a conduit during a procedure, comprising:
placing the apparatus, wherein the apparatus comprises a frame and a membrane into the conduit;
filtering a fluid in the conduit utilizing the membrane to prevent the debris from passing a proximal side of the membrane;
compressing a proximal end of the frame, the frame connected to the membrane at a distal end; wherein the distal end of the frame extends the membrane to the walls of the conduit; and wherein the membrane remains extended during the removal of the apparatus from the conduit.

11. The method of claim 10, further comprising filtering a fluid in the conduit using the frame.

13. The method of claim 10, further comprising enclosing a debris in the apparatus using the frame to prevent the debris from passing the proximal side of the membrane.

14. The method of claim 10, wherein the conduit is an artery.

15. An internally disposed apparatus for inside a fluid conduit during a medical procedure, comprising:
a filtering membrane; and
a frame connected to the membrane at a distal end and extended to hug the walls of the fluid conduit, the frame to be compressed at a proximal end while the distal end remains extended;
wherein the distal end of the frame extends the membrane to the walls of the fluid conduit; and
wherein the membrane remains extended during the removal of the apparatus from the fluid conduit.

16. The internally disposed apparatus for inside a fluid conduit during a medical procedure of claim 15 further comprising:
a rod; and,
a guidewire.

17. The internally disposed apparatus for inside a fluid conduit during a medical procedure of claim 16 wherein the rod comprises a 0.35 mm diameter.

18. The internally disposed apparatus for inside a fluid conduit during a medical procedure of claim 16 wherein the guide wire comprises a 0.018 mm guidewire.

19. The internally disposed apparatus for inside a fluid conduit during a medical procedure of claim 16 wherein the rod comprises a 0.35 mm diameter comprises a diameter ranging from 0.01 mm to 15 mm.

20. The internally disposed apparatus for inside a fluid conduit during a medical procedure of claim 15 wherein the filtering membrane is housed within a sheath during insertion.

21. The internally disposed apparatus for inside a fluid conduit during a medical procedure of claim 20 wherein the sheath is used during the procedure as a suction or flushing device in order to remove debris from the filter membrane.
